(19) United States
(12) Patent Application Publication
(10) Pub. No.: US 2021/0310073 A1
(43) Pub. Date: Oct. 7, 2021

(54) A GENETIC PREDISPOSITION TO LIVER DISEASE

(71) Applicant: Baylor College of Medicine, Houston, TX (US)

(72) Inventor: Richard N. Sifers, Houston, TX (US)

(21) Appl. No.: 17/272,187

(22) PCT Filed: Sep. 6, 2019
(86) PCT No.: PCT/US2019/049976

§ 371 Date: Feb. 26, 2021

Related U.S. Application Data
(60) Provisional application No. 62/727,853, filed on Sep. 6, 2018.

(51) Int. Cl. C12Q 1/6883; C12Q 1/6827

(52) U.S. Cl. CPC C12Q 1/6883; C12Q 2600/156; C12Q 1/6827

(57) ABSTRACT

Embodiments of the disclosure concern a genetic signature for individuals suspected of having liver disease, including end stage liver disease. In particular embodiments, the genetic signature comprises two different genetic markers, including a polymorphism and differential probe hybridization. In specific embodiments, the polymorphism is the rs4567 SNP in the 3′ UTR of the MAN1B1 and the copy number variation is in chromosome 22.

This application claims priority to U.S. Provisional Patent Application Ser. No. 62/727,853, filed Sep. 6, 2018, which is incorporated by reference herein in its entirety.

### TECHNICAL FIELD

Embodiments of the disclosure include at least the fields of molecular biology, cell biology, genetics, diagnostics, and medicine.

### BACKGROUND

Protein conformational diseases represent a classification of diseases in which the pathology emerges from the toxic of a protein that fails to correctly fold after biosynthesis in cells. These diseases include cystic fibrosis, many lysosomal storage diseases, osteogenesis imperfecta, hypercholesterolemia, hereditary hemochromatosis, Parkinson's Disease, Alzheimer's Disease, Wilson Disease, liver and lung diseases associated with serum alpha1-antirypsin deficiency, for example. In some of these diseases, a misfolded protein accumulates at the site of biosynthesis and is toxic. To guard against the toxic accumulation of aberrant proteins, systems operate in cells to selectively identify and target misfolded proteins for intracellular destruction. An additional system operates at the tissue/organ level to promote cell proliferation as a means to replace potentially damaged cells. A current challenge in biomedical research is to characterize the repertoire of different cellular protein disposal and cell proliferation systems that contributes to organ health. Likewise, a current interest is to elucidate how the genetic disruption of these systems contribute to the molecular pathogenesis, and/or phenotypic variability, of the aforementioned numerous disorders. The resulting molecular insight is useful to generate more accurate prognostic indicators and drive the development of effective therapeutic interventions for the management of specific pathologic complications.

Alpha1-antitrypsin (AAT) is a serine proteinase inhibitor (SERPIN) that functions as a potent proteinase inhibitor. Normal circulating concentrations are sufficient to prevent the destruction of lung elastin fibers. Nevertheless, many naturally occurring genetic variants of AAT exist in which an amino acid substitution or premature truncation of the polypeptide prevents the acquisition of native structure following biosynthesis in the hepatocyte endoplasmic reticulum (ER). Rather than secreted into the bloodstream, these improperly-folded proteins are retained in cells and are subjected to intracellular proteolysis.

Although the non-native AAT monomers are subjected to proteasomal degradation by a mechanism designated “ER Associated Degradation” (ERAD), for some genetic variants, like variant ATZ, a competing fate is polymerization (FIG. 1). Several mechanisms of polymerization have been proposed, but the consensus is that the accumulated polymers are cytotoxic and cause liver injury in both humans and transgenic mouse models. Currently, orthotopic liver transplantation is the preferred treatment for the end-stage liver disease in humans. Because ATZ polymers accumulate in inclusion bodies, autophagy functions as a slow process that clears these inclusion bodies from cells. In terms of developing novel therapies, specific autophagy enhancers are being tested as a potential approach to remove the toxic species (Hidvegi T et al., 2010). In addition, the RNAi-mediated abrogation of ATZ expression represents an even more novel treatment (Turner A M et al., 2018). Although these corrective strategies are in demand, a reliable prognostic indicator capable of identifying patients who are predisposed to develop severe liver disease is currently lacking in the art but is needed.

### BRIEF SUMMARY

The present disclosure is directed to systems, methods, and compositions that identify an individual as having liver disease or having an increased risk of having liver disease compared to the average individual in the population. In particular embodiments, the disclosure encompasses methods for determining when an individual has or is at an increased risk of having liver disease. The method may include detection of the presence of one or more particular nucleotides of a polynucleotide, such as in a gene. The one or more particular nucleotides may be in a coding region or a non-coding region of a gene. In specific cases, the nucleotide is a single nucleotide polymorphism (SNP). In some embodiments, when the presence of a particular nucleotide is detected, the method(s) used to detect the nucleotide may include selective oligonucleotide probes, arrays, allele-specific hybridization, molecular beacons, restriction fragment length polymorphism analysis, polymerase chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. Methods of the disclosure include those that seek the identity of a particular nucleotide at a particular position regardless of the outcome of the analysis. In particular embodiments, the one or more particular nucleotides is a SNP, and in specific cases the SNP being detected is the rs4567 SNP, which is located in the 3′ UTR of the MAN1B1 gene. Methods for non-human animals may include analysis of any ortholog of MAN1B1. When the particular nucleotide being detected at this location is an adenosine, the individual may have liver disease or may be at an increased risk of having liver disease.

In certain embodiments, the method analyzes differential probe hybridization of an allele. Although the differential probe hybridization may be for any reason, in some cases the differential hybridization is related to the copy number of an allele, including a particular allele. In particular embodiments, when the method analyzes differential probe hybridization of an allele, the method used to do so is comparative genomic hybridization. In specific embodiments, the differential probe hybridization is a measure of the copy number of an allele, the method(s) used to analyze the copy number may include fluorescent in situ hybridization, comparative genomic hybridization, arrays, polymerase chain reaction, sequencing, or a combination thereof, for example.

In particular embodiments, the methods involve (1) analyzing a polymorphism, and (2) detecting differential probe hybridization of an allele in the individual, and in specific cases the polymorphism and the differential probe hybridization reside in different loci of the genome of the individual. In such embodiments, the methods of detection and/or analysis include the methods described herein for detecting and/or analyzing the polymorphism or differential probe hybridization individually, in some cases. Particular embodiments of the disclosure require both analysis for a particular polymorphism and analysis for a particular differential probe hybridization, and the output of both of these steps may be utilized in determining whether or not an individual has liver disease or is at risk for having liver disease, including end-stage liver disease.

In particular embodiments, the individual having, or at risk of having, liver disease may have an alpha1-antitrypsin deficiency. In some embodiments, the individual has a variant of the alpha1-antitrypsin gene. The variant is the ATZ variant, in some cases, wherein the individual is homozygous for the ATZ variant (Z/Z), heterozygous for the ATZ variant and another non-ATZ variant (Z/+), or homozygous for a non-ATZ variant (+/+).

The individual, after having been determined to have liver disease or have an increased risk of liver disease may be provided one or more treatments based on the determination. The treatment(s) could be any treatment intended to improve liver function in the individual including vitamin supplementation, liver transplantation, carbamazepine, gene therapy, cell therapy, antisense therapy, RNA interference, or a combination thereof, for example.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1. Competing fates for newly synthesized alpha1-antitrypsin. The newly synthesized nonnative alpha1-antitrypsin polypeptide (aat) is translocated into the endoplasmic reticulum (ER) (step 1) where it can correctly fold (step 2) and undergo secretion. Misfolded molecules are subjected to proteasomal degradation (step 3). MAN1B1 is known to play an essential role in targeting this population for proteasomal degradation. Specific genetic variants of nonnative aat, like AAT-Z (ATZ), can cause the unfolded monomer to undergo spontaneous polymerization (step 4), which are slowly eliminated by autophagy. The accumulated polymers are capable of promoting cytotoxicity, liver injury, and cirrhosis. Step 2 and the step from AAT to secretion reflect aspects of AAT conformation maturation. Step 3 leads to intracellular degradation.

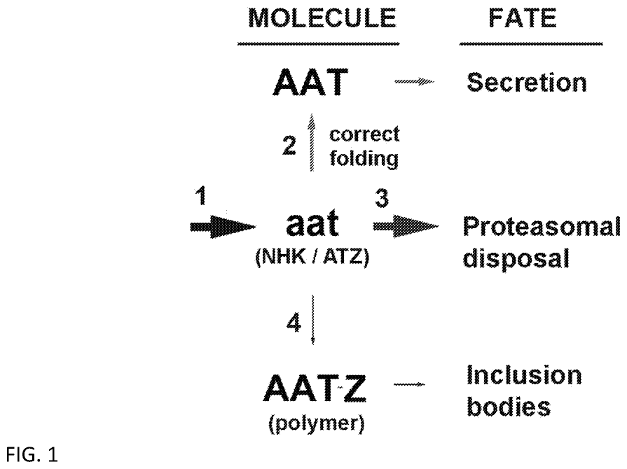

FIGS. 2A-2D. Validation of miR-362-5p involvement. 2A. ATZ-mediated induction of endogenous miR-362-5p in transfected HeLa cells. HeLa cells were mock-transfected with empty vector (mock) or with a recombinant ATZ expression vector (ATZ). ATZ was detected by western blotting of the lysed cells. The relative concentration of miR-362-5p in equivalent cell aliquots is depicted (four replicate experiments −/+ one stnd deviation). 2B. The miR-362-5p mimic preferentially suppresses the concentration of transfected MAN1B1 (A allele) in HeLa cells. HeLa cells were transfected with either an expression vector that encodes recombinant MAN1B1 from the “A” allele or “G” allele at rs4567. Equivalent transfection efficiencies were validated by the parallel analysis of cell extracts. Transfection was accompanied by the addition of equal concentrations of either a control (Ctl) miRNA or the miR-362 (362) mimic. Relative changes in the human recombinant MAN1B1 concentration after 48 hours was performed by quantitative blotting from equivalent cell aliquots represented as a bar graph (four replicate experiments −/+ one stnd deviation). 2C. Effect of the miR-362-5p mimic on the concentration of endogenous MAN1B1 in genotype-specific fibroblasts JT002 and JT008. Cell lines were grown to confluency in duplicate dishes and subjected to mock treatment, or treated with equal concentrations of a Non-Specific (N-S) miRNA or the miR-362 mimic. Western blot analyses from cell extracts detected endogenous MAN1B1 and actin in duplicate dishes. The concentration of endogenous MAN1B1 was diminished >60% with miR-362-5p. 2D. Patient-derived Z/Z fibroblasts (ZZ fib), the genotype at rs4567, and the effect of the miR-362 mimic on the concentration of the endogenous MAN1B1 concentration are shown. NC=no change and the arrow represents a consistent reduction of >50% from >4 replicate experiments.

FIG. 3. One embodiment for the induction of miR-362 by stress induced from the expression of the ATZ protein. Two microRNAs are generated (5p and 3p). 5p binds a new microRNA binding site on the gene for Man1b1, thereby suppressing its capacity to promote the degradation of ATZ. In contrast, 3p is known to bind a specific site in the downstream 3′-UTR for the mRNA transcript of Erbb2 (TOB2), suppressing its expression, and allows cell proliferation to perform a corrective function at the tissue/organ level. However, a CNV in the transcription start site for the TOB2 gene blunts cell proliferation. The two genomic defects (generating a pathologic genomic signature) are underlined. The effect on liver pathology is shown in boxes.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “allele” refers to one of at least two variants, forms, or mutations of a gene, wherein the gene is at the same position on a chromosome.

As used herein, the terms “arrays”, “microarrays”, and “DNA chips” refer to an array of distinct oligonucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. The oligonucleotides on the array may be designed to bind or hybridize to specific nucleic acids, such as a specific SNP or a specific CNV, for example.

As used herein, the term “ATZ”, “ATZ variant”, “Z variant”, or “Z” refers to the ATZ allele of the alpha1-antitrypsin. This mutant form of alpha1-antitrypsin typically leads to alpha1-antitrypsin protein misfolding.

As used herein, the term “genetic signature” refers to one or more features for comparing a particular genome to another genome or a reference genome. In specific embodiments, the features may comprise one or more SNPs, CNVs, insertions, deletions, repeats, duplications, transpositions, inversions, substitutions, recombinations, mutations, chromosomal rearrangements, chromosomal duplications, chromosomal deletions, homozygosity of one or more alleles, heterozygosity of one or more alleles, or a combination thereof. In specific embodiments, the genetic signature may comprise, consist of, or consist essentially of one or more SNPs and/or one or more CNVs.

As used herein, the term “copy number variation” or “CNV” is a duplication or deletion of a DNA segment encompassing a gene, genes, segment of a gene, or DNA region regulating a gene, as compared to a reference genome. In some embodiments, a CNV is determined based on variation from a normal diploid state. In some embodiments, a CNV represents a copy number change involving a DNA fragment that is 1 kilobase (kb) or larger.

The term “differential probe hybridization” as used herein is defined as a detectable increase in the binding of one or more overlapping probes at a specific DNA locus that is detected as a reproducible (at least) three-fold enhancement of signal intensity.

As used herein, the term “oligonucleotide” refers to a short chain of nucleic acids, either RNA, DNA, and/or PNA. The length of the oligonucleotide could be less than 10 base pairs, or at minimum or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs. The oligonucleotide can be synthesized using by methods including phosphodiester synthesis, phosphotriester synthesis, phosphite triester synthesis, phosphoramidite synthesis, solid support synthesis, in vitro transcription, or any other method known in the art.

As used herein, the term “PCR primer” refers to an oligonucleotide that is used to amplify a strand of nucleic acid in a polymerase chain reaction (PCR). Primers may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100% homology to the template the primers hybridize to, wherein the 3′ nucleotide of the primer is complementary to the template. In some embodiments, lower annealing temperatures are used for initial cycles, for example cycles 1, 2, 3, 4, and/or 5, of the reaction.

As used herein, the term “polymorphism” refers to a genetic variant in a population. Further, a “single nucleotide polymorphism” also known as a “SNP” refers to a genetic variant arising from the change of one single nucleotide in a genome.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Methods of Use

In particular embodiments, the disclosure concerns a genetic signature that, when detected in an individual, determines that the individual has or is at an increased risk of having liver disease compared to the average individual in the population. The information of the method may be utilized as a diagnosis, in certain embodiments. In some embodiments, the genetic signature being detected includes determining the identity of a particular nucleobase at a specific single nucleotide polymorphism (SNP). The SNP may be the SNP at location rs4567 in at least some cases. The SNP at location rs4567 is defined herein at position 101 in SEQ ID NO:1. Detection of an A nucleotide at that particular SNP is an indicator that the individual has, or has an increased risk of having, liver disease. In some embodiments the genetic signature being detected comprises differential probe hybridization of an allele. This differential probe hybridization may be detected by the nucleic acid sequence represented by or comprising all or some of SEQ ID NO:2. The differential probe hybridization may concern the entirety of SEQ ID NO:2, or may be at least 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, or 99% of the span of nucleotides in SEQ ID NO:2, including contiguous nucleotides. The CNV differential probe hybridization may be at least 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100% homologous to SEQ ID NO:2.

In cases wherein the method includes determining identity of the SNP at location rs4567 and determining whether there is differential probe hybridization at part or all of SEQ ID NO:2, the order of the determining steps may be in any order.

In particular embodiments, the genetic signature being detected in an individual comprises both a particular nucleobase at a specific SNP and the copy number variation of an allele. In particular embodiments, the individual suspected of having liver disease, including end-stage liver disease, has an alpha1-antitrypsin deficiency. The individual may have at least one copy of the ATZ variant of the alpha1-antitrypsin gene, leading to alpha1-antitrypsin deficiency. Individuals with alpha1-antitrypsin deficiencies, including individuals with at least one copy of the ATZ variant, often develop lung and liver pathologies. However, not all of the individuals develop liver pathologies. In particular embodiments of the disclosure, the detection of the genetic signature in an individual, including individuals with at least one copy of the ATZ variant, indicates the presence or increased risk of the presence of liver disease in the individual; in some embodiments the method includes a therapeutic intervention to treat the individual. In some embodiments, the therapeutic intervention includes liver transplantation or one or more other therapies that improve liver function in individuals with liver disease. In certain embodiments, when an individual is determined to have an alpha1-antitrypsin deficiency, they are subject to one or more certain methods of this disclosure to detect if the individual has a certain genetic signature. In specific embodiments, an individual suspected of having an alpha1-antitrypsin deficiency is also subject to method(s) of the disclosure.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position, reference is generally made to the protein-encoding strand, only for the purpose of convenience.

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, polymerase chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. In some embodiments the method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

Particular embodiments concern the methods of diagnosing and/or treating liver disease in an individual. In specific embodiments, the methods for diagnosing and/or treating liver disease comprise obtaining a sample from the individual, detecting a genetic signature in the individual, and providing a therapeutic intervention to the individual. Particular embodiments involve obtaining a sample from an individual, which may include obtaining a blood and/or plasma, for example. The biological sample may be a tissue sample, such as a cheek, skin, or hair sample, a biopsy, or any tissue sample that was taken or derived from the individual, wherein the sample may be used to obtain genetic material from the individual.

### III. Single Nucleotide Polymorphism Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, polymerase chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP. One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR. In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### IV. Copy Number Variation Detection

Particular embodiments of the disclosure related to differential probe hybridization concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array, wherein the array is capable of detecting CNVs on the entire X chromosome and/or all targets of miR-362. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using qPCR can be performed in the same manner as described elsewhere in this disclosure. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used comprise whole genome sequencing, whole exome sequencing, and/or targeted sequencing.

### V. Differential Probe Hybridization

In particular embodiments, an individual is determined to have liver disease or have an increased risk of liver disease compared to an average individual in the population. In particular embodiments, this may employ standard genome hybridization methods, such as the Comparative Genome Hybridization Array technique. In studies by the inventor, genomic DNA extracted from individuals who exhibit liver disease consistently exhibit at least a 2-fold or 3-fold increase in probe hybridization at the TOB2 gene promoter (for example, a region in SEQ ID NO:2), as compared to individuals in which the disease never emerged. The fold increase may be at least a 3, 4, 5, or 6-fold or more increase in probe hybridization at part or all of SEQ ID NO:2.

In one example, three overlapping probes were used for differential detection at SEQ ID NO:2. Whole genome sequence comparison demonstrated that the hybridizing genomic DNA locus (CGCAGGGTTGCTAAGGGTGAAACTTTTCATTGACTTT; SEQ ID NO:3) is not duplicated in any other part of the human genome. SEQ ID NO:3 is the genomic locus to which the three examples of probes were able to hybridize at least in part. In specific embodiments, differential probe hybridization encompasses binding of one or more probes at part or all of SEQ ID NO:3 in the genome of an individual being tested for liver disease or a risk thereof. The probe(s) may bind to at least at 37, 36, 35, 34, 33, 32, 31, 30, 29, 28, 27, 26, 25, 24, 23, 22, 21, 20, or fewer contiguous nucleotides of SEQ ID NO:3.

| TOB2 Sequence: | |
|---|---|
| FS: | 5′-CGGCCCGAAGTGAGCAAAGTCAATGAAAAGTTTCACCCTTAGCAACCCTGCGCA-3′ |
| RS: | 3′-GCCGGGCTTCACTCGTTTCAGTTACTTTTCAAAGTGGGAATCGTTGGGACGCGT-5′ |
| Probes: | |
| FS-1: | 5′-CGGCCCGAAGTGAGCAAAGTCAATGAAAAG-3′ |
| FS-2: | 5′-TGAAAAGTTTCACCCTTAGCAACCCTGCGCA-3′ |
| RS | 3′-GCCGGGCTTCACTCGTTTCAGTTACTTTTCA-5′ |

An example of differential hybridizing probes are provided above. The forward strand (SEQ ID NO:4) and reverse strand (SEQ ID NO:5) of the example of the TOB2 hybridizing sequence is shown on top. Two forward strand (FS) probes are shown, as well as the Reverse Stand (RS) probe. The orientation of all DNA sequences are also shown. FS-1 is SEQ ID NO:6; FS-2 is SEQ ID NO:7; and RS is SEQ ID NO:8.

The probes to be utilized for differential probe hybridization may be of any length, including at least 20 nucleotides and/or no more than 40 nucleotides. In specific cases, the probes are at least or no more than 20, 21, 22, 23, 24, 25, 26, 27, 28, 29, 30, 31, 32, 33, 34, 35, 36, 37 38, 39, or 40 or more nucleotides in length.

In particular embodiments, the differential probe hybridization at or around part or all of SEQ ID NO:2 provides information of individuals that have liver disease or have an increased risk for liver disease compared to the average population. The differential probe hybridization in this region may be for any reason. In specific embodiments, the differential probe hybridization is at least in part from one or more of the following: (1) a copy number variation at this locus; (2) differential methylation of cytosines (which is common epigenetic mark at many gene promoter regions); or (3) differential methylation of adenines (an epigenetic regulator of gene expression). Each of these mechanisms could account for an increased probe hybridization and in specific embodiments, also an elevation of the TOB2 mRNA transcript. Without being limited by theory, in specific embodiments, the increased probe binding allows expression of the TOB2 mRNA transcript to increase to an extent that prevents the 3p microRNA lowering of the TOB2 mRNA. This impairment directly or indirectly prevents the proliferation of healthy hepatocytes, in specific embodiments.

### VI. Liver Disease and Alpha1-Antitrypsin Deficiency

Particular embodiments of the disclosure concern determining whether or not an individual has or is at risk for liver disease. Liver disease may include chronic liver disease, end stage liver disease, cirrhosis, chronic liver failure, liver fibrosis, hepatitis, cryptogenic cirrhosis, malignant hepatoma, and so forth. Additionally, liver disease could include the conditions of the liver comprising inflammation, tumors, metabolic disorders, alcoholic liver disease, non-alcoholic fatty liver disease (NAFLD), viral hepatitis, NASH, or a combination thereof. Liver disease symptoms may comprise jaundice, swelling or edema, nausea and/or fatigue, abdominal pain, and a combination thereof, for example. In particular embodiments, an individual is at risk of liver disease because of an alpha1-antitrypsin deficiency. Alpha1-antitrypsin deficiency is a disorder that typically causes lung disease in individuals between the ages of 20 years old to 50 years old. However, a portion of individuals with alpha1-antitrypsin deficiency develop liver disease. In these individuals, alpha1-antitrypsin can be misfolded after synthesis in the liver resulting in accumulation in the liver, which leads to liver disease. In particular embodiments, the genetic signature detected by the methods described herein identifies individuals who will develop liver disease, or who are at an increased risk to develop liver disease. In embodiments of the disclosure, individuals with alpha1-antitrypsin deficiency who have the genetic signature encompassed herein has liver disease, will have liver disease, or are at an increased risk for having liver disease. An individual having an increased risk for having liver disease may be an individual with a greater risk for liver disease when compared to the general population.

In particular embodiments of the disclosure, diagnosing an individual that has liver disease, will have liver disease, or that is at an increased risk of having liver disease indicates the need for therapeutic or prophylactic intervention. The therapeutic or prophylactic intervention may include any method of restoring liver function at least in part, including, vitamin supplementation, liver transplantation, carbamazepine, gene therapy, cell therapy, antisense therapy, RNA interference, and a combination thereof.

Particular embodiments of the disclosure concern methods of treating an individual having or at an increased risk of having liver disease, including end stage liver disease. The methods to treat an individual include therapeutic or prophylactic interventions and may be vitamin supplementation, liver transplantation, carbamazepine, gene therapy, cell therapy, antisense therapy, RNA interference, and a combination thereof. The method of using therapeutic or prophylactic intervention may be because of the detection of a genetic signature in the individual determined by the methods described herein.

In some cases, the pathologic genetic signature of the disclosure is related to the development of liver disease in individuals who have been diagnosed with alcoholic liver disease, non-alcoholic fatty liver disease (NAFLD), viral hepatitis, NASH, etc. whether or not these individuals also exhibit a single Z allele. Specifically, in some embodiments a single Z allele can be related to liver disease in any individual if they also exhibit the pathogenic genetic signature.

### VII. Detection Kits and Systems

One can recognize that based on the methods described herein, detection reagents, kits, and/or systems can be utilized to detect the SNP and/or the differential probe hybridization related to the genetic signature for diagnosing an individual (the detection either individually or in combination). The reagents can be combined into at least one of the established formats for kits and/or systems as known in the art. As used herein, the terms “kits” and “systems” refer to embodiments such as combinations of at least one SNP detection reagent, for example at least one selective oligonucleotide probe, and at least one hybridization detection reagent and/or mechanism, for example at least one oligonucleotide. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

In specific embodiments, the kit may comprise one or both of a composition for detecting a polymorphism and a composition for detecting differential hybridization, including CNV. In certain embodiments, the polymorphism detected is polymorphism rs4567 as represented by position 101 of SEQ ID NO:1. In certain embodiments, the differential hybridization occurs at or around SEQ ID NO:2. The composition in the kit for detecting the polymorphism may be selected from the group consisting of one or more oligonucleotides, one or more primers suitable for amplifying the polymorphism, one or more sequencing reagents, and a combination thereof, for example. The composition(s) in the kit for detecting differential hybridization may be selected from the group consisting of one or more primers suitable for amplifying a region of SEQ ID NO:2, one or more sequencing reagents, one or more arrays, and a combination thereof. Any composition in the kits may or may not be labeled for detection of any kind, including by colorimetry or fluorescence, for example.

### VIII. Examples

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### A SNP in the Gene for ERMan1/MAN1B1 Functions as a Genetic Modifier of Early-Onset End-Stage Liver Disease

The accumulation of insoluble ATZ polymers within inclusion bodies derived from the hepatocyte secretory pathway is the leading genetic cause of childhood liver disease in the United States. However, its clinical presentation among homozygous Z/Z patients is highly variable. Considering this variability, studies were sought to identify genetic modifiers of the liver disease, and then mechanistically analyze the underlying abnormality with respect to the management of accumulated ATZ within the secretory pathway.

Because the newly synthesized monomer functions as the direct precursor of toxic ATZ polymers, studies were performed to investigate whether a hindered rate of monomer degradation might influence the age-at-onset of the liver disease. In support of this general notion, one group reported that a quantifiable delay in the degradation of the ATZ monomer occurs in transduced fibroblasts and those differentiated into hepatocytes derived from patients who eventually underwent liver transplantation, as compared to those who exhibit normal liver function (Wu Y et al., 1994).

The intracellular concentration of human ERMan1/MAN1B1 plays both a deterministic and rate-limiting role (Wu Y et al., 2003) to regulate entrance of the nonnative AAT monomer into ERAD. The sequencing of MAN1B1 exons in genomic DNA from Caucasian Z/Z patients (obtained from the University of Minnesota) led to the identification of a disease modifier. Statistical analyses identified that homozygosity for a single-nucleotide polymorphism (SNP) in the 3′-untranslated region of the MAN1B1 gene (i.e., the A allele for rs4567) coincides (P<0.0003) with the development of end-stage liver disease in Z/Z individuals under two years of age (Pan S et al., 2009). In that same study, additional functional assays demonstrated that the A allele is responsible for generating a conditional MAN1B1 hypomorphic allele capable of suppressing the translation of MAN1B1 in response to ER stress caused by the intracellular accumulation of ATZ (Pan S et al., 2009).

### Example 2

### Involvement of miR-362-5p

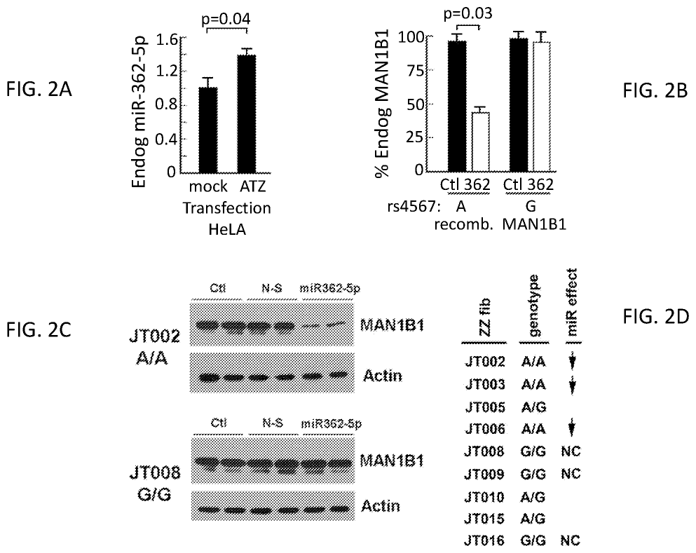

Because rs4567 is located in the 3′-UTR of the mature RNA transcript for ERMan1/Man1b1, an experimental strategy to functionally validate the findings of Example 1 was performed and generated a new mechanistic understanding (FIG. 2). Because microRNAs (miRNA) are known to bind the 3′-UTR of mRNAs and suppress the translation of the encoded protein, miRNA dependent suppression of Man1b1 translational is a potential mechanism. In addition, there are already identified microRNA binding sites on the mRNA that encodes ERMan1/Man1b1 (Pan S et al., 2013). For the experimental approach, a combination of predictive algorithms, miRNA profiling, and mammalian cell expression studies were employed. The information predicted that the A allele of rs4567 is responsible for generating a new binding site for miR-362-5p. Subsequently, it was established that this miRNA is induced in HeLa cells response to the expression of ATZ (P=0.04), whereas another predicted miRNA was not induced (FIG. 2A). Importantly, the same observation was made when ATZ was replaced by another misfolded AAT variant that does not polymerize (not shown), indicating that the AAT monomer is responsible for promoting cell stress. Moreover, the addition of a commercially-available miRNA mimic was able to specifically suppress the translation of recombinant MAN1B1 having the A allele at rs4567 in transfected HeLa cells (P=0.03), but not for Man1b1 that bears the G allele at rs4567 (FIG. 2B). Finally, it was determined that the endogenous Man1b1 protein present in genotyped Z/Z patient fibroblasts was diminished in patients who were homozygous for the A allele at rs4567, but not in those individuals who were homozygous for the G allele at rs4567 (FIGS. 2C & D). Based on these numerous lines of experimental evidence, it was concluded that the SNP generated a conditional hypomorphic allele that, when inherited in a homozygous fashion, can impair a cell's capacity to efficiently degrade the ATZ monomer, possibly leading to an accelerated formation of toxic monomers.

### Example 3

### Identification of the Central Defect in the Gene for TOB2

A subsequent set of experiments analyzed the nucleotide present at rs4567 in genomic DNA samples from the Alpha1 Tissue and DNA Bank at the University of Florida. Importantly, it was discovered that homozygosity for the rs4567 A allele exists in numerous Z/Z patients who never developed any detectable liver disease. This led to the conclusion that another pathologic factor likely exists. Importantly, this understanding supports the conclusion that rs4567 merely represents a modifier, rather than the causal defect that promotes severe liver cirrhosis.

To follow-up on this discovery, it was determined whether the expression of miR-362 (the parental form of miR-362-5p) must be elevated to induce a pathological effect. The approach was to perform a Copy Number Variation (CNV) study on patient genomic DNA. Because miR-362 is encoded by a gene on the human X chromosome, experts at Agilent/SureDesign developed a microArray that would detect CNVs on the entire X chromosome, plus additional genomic loci that have been reported to serve as targets for miR-362. The analysis was performed on genomic DNA samples from patients that were homozygous for the A allele at rs4567. One group represented patients who had developed end-stage liver disease. The other groups were patients who had no detectable liver abnormalities during their lifetime. The analyses were successful in that numerous CNVs were detected as either increases, decreases, or deletions in genomic copy number in chromosomes from all samples. However, one elevated CNV was consistently detected in all the samples from diseased patients, but none from the control group (without disease). The detected CNV was on human chromosome 22 in the gene for TOB2 which is a known target for miR-362-3p (Shen H et al., 2015).

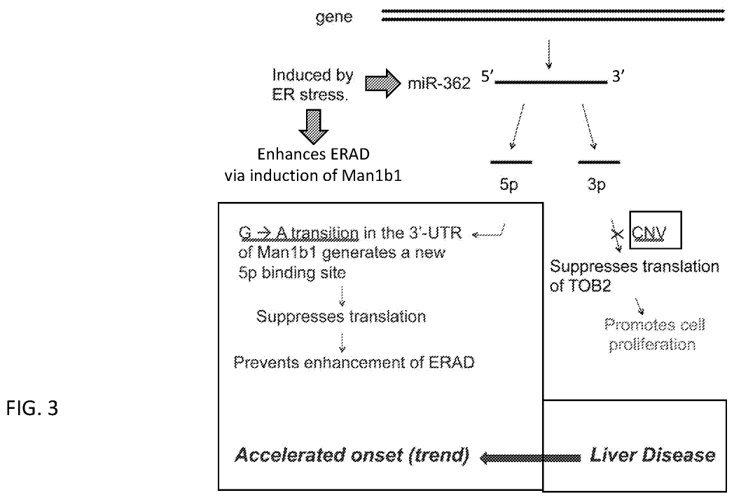

This is a very important observation because TOB2 is a translational suppressor that functions to hinder cell proliferation. It is the binding of miR-362-3p that functions to release this block as a mechanism to induce cell proliferation (Shen H et al., 2015). In the proposed model, the CNV detected functions to hinder the binding of miR-362-3p and therefore blocks the induction of hepatocyte proliferation (FIG. 3). This in turn, greatly compromises the liver's capacity to utilize hepatocyte proliferate as a normal corrective mechanism. Taken together, there is now an identified single nucleotide polymorphism (SNP) plus a copy number variation (CNV) at distinct loci in the human genome (i.e., Man1b1 and TOB2) that is responsible for promoting the development of end-stage liver disease in patients who exhibit alpha1-antitrypsin deficiency.

Importantly, these predictions are consistent with reports in the literature in which ATZ degradation is suppressed in cells from patients who exhibit severe liver disease (Wu Y et al., 2003). Also, liver hepatocyte proliferation is normally accelerated in response to ATZ expression in the transgenic PiZ mouse model (Rudnick D A et al., 2004) where the animals fail to develop significant liver injury. In fact, this phenotype led to the pursuit of the identification of causal agent for the severe liver disease. Finally it should be noted that a hallmark of severe liver disease in patients is accompanied by a greater-than-normal abundance of ATZ-bearing hepatocytes in the liver which is consistent with failures in both protein degradation and cell proliferation (Lindblad D et al., 2007).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### IX. Sequences

SEQ ID NO: 1
TGTGGGCCGACCAGAGGGGGGCTTCGAGGTGGTCCCTGGTACTGGGGT
GACCGAGTGGACAGCCCAGGGTGCAGCTCTGCCCGGGCTCGTGAAGCC
TCAGRTGTCCCCAATCCAAGGGTCTGGAGGGGCTGCCGTGACTCCAGA
GGCCTGAGGCTCCAGGGCTGGCTCTGGTGTTTACAAGCTGGACTCAGG
GATCCTCCT

(where the underlined R is the SNP position, wherein the underlined R represents either an A nucleotide or a G nucleotide)

| SEQ ID NO: 2: | |
|---|---|
| aaggagtgcg gcgcggcgct gtgacaggga ggggaagacg gggagccgga | 40172948 |
| cgcacgaccg gcgagcacag aagggcacag gagctgctcc gcgagatacc | 40172898 |
| agcgagctga ggcgcggggc tggctgggaa gtagtcccgg ccgggccctg | 40172848 |
| gccccgcccc tgcagggcgg agggacgcat cacgcagggg tggggaagcc | 40172798 |
| ggatccgtgc gcagggttgc taagggtgaa acttttcatt gactttGCTC | 40172748 |
| ACTTCGGGCC GGGGCGCGGG AAGGTGCGGG TTGTTGGCTG AGGCAAGCCG | 40172698 |
| TGGCTCGGGC GACGCGGGGC CAGGAGGGCA GGACCGTCTG CCGGCGACAG | 40172648 |
| CCCATACCGT CTGCCGTTGC GCGACACGAA CCACCCTCCG ATCCGCCATC | 40172598 |
| CCCGCGTCGT CGCCGCTAGT CCGCCCGCCC GCCCTCGGGC CCCCCGCTGC | 40172548 |
| CGAGCCCGAC CTCCTAAGAG CTGAAAGAAA TTATTGAGAG TCATAGTCCA | 40172498 |
| TAGCCCCCTG CTTCGTCCCC CAACCCTCAA CGACGAAAAG GACTTCGGTC | 40172448 |
| CCCTGGCCCG GCGACGCCCG GGAAGGAAAG GAGAGCGACC TCCGCCCCGC | 40172398 |
| GCTCAGGCCA CCCTGGAGGG AGAAGCCGCC CCGCGGCCGG TAGAGCGCCC | 40172348 |
| CGCCGCCGCG GAGACCCGAG TGAGTGAgtg ggggggcgtg cgggcggggg | 40172298 |
| gcccgccccg agacccagcc ggccggacta cctcttccac cccgccccac | 40172248 |
| tcccgctttg ctggactctc cacgttgctg ccgctccctc cccgcctggc | 40172198 |
| cagcattttc tttccccatc cagcttccct ccagacccca ctttccccag | 40172148 |
| ctcctcttct cacgccatct gtgctcctgg ggtgcccaag agctcctttt | 40172098 |
| tctcccagtt ccaagagtgg ccctctcctg ggtggggtgc atttcctccc | 40172048 |

Human TOB2 gene sequences. Numbers present on the right of each line represent the nucleotide coordinates (reverse strand) in human Chromosome 22 (NCBI36/hg18 Assembly). The initial lower case nucleotides represent 5′ upstream sequence. Capital letters designate the first exon (comprised entirely of 5′ untranslated nucleotides). The last sequence of lowercase letters represents part of the first intron. The nucleotide sequence shown in bold type represents the locus that exhibits an enhanced (for example, at least 2-fold or 3-fold) probe hybridization over non-diseased individuals. The bolded sequence includes 5′-upstream sequences, the TOB2 transcription start site, and some of the first exon.

1. A method for diagnosing an individual as having liver disease or having an increased risk for liver disease, comprising:
(a) analyzing a region of nucleic acid from an individual suspected of having liver disease or an increased risk thereof, wherein said nucleic acid includes polymorphism rs4567 as represented by position 101 of SEQ ID NO:1, to detect the presence or absence of an A nucleobase at position 101 of SEQ ID NO:1;
(b) determining differential probe hybridization of an allele represented by part or all of SEQ ID NO:2; and
(c) diagnosing said human as having an increased risk for liver disease when the analysis in step (a) detects the presence of an A nucleobase at position 101 of SEQ ID NO:1 and there is at least three-fold differential probe hybridization at the allele represented by SEQ ID NO:2 as determined in step (b).

2. The method of claim 1, wherein the method for analyzing the region of SEQ ID NO:1 in (a) comprises the steps of:
(1) amplifying a region of nucleic acid from said individual that includes polymorphism rs4567 as represented by position 101 of SEQ ID NO:1 or its complement thereof to thereby generate an amplicon comprising said polymorphism;
(2) testing for said polymorphism rs4567 by contacting said amplicon with an oligonucleotide that selectively hybridizes to A at said position 101 of SEQ ID NO:1 or T at said complement; and
(3) detecting the presence of said A or said T.

3. The method of claim 2, wherein said oligonucleotide is an allele-specific probe.

4. The method of claim 2, wherein said oligonucleotide comprises a segment of said SEQ ID NO:1 or its complement thereof, wherein said segment comprises at least 12 contiguous nucleotides in length and includes said position 101 or its complement.

5. The method of claim 2, wherein said oligonucleotide is detectably labeled with a fluorescent label.

6. The method of claim 1, wherein said individual is homozygous for said A or said T.

7. The method of claim 1, wherein said individual is heterozygous for said A or said T.

8. The method of claim 1, wherein the sequence represented by SEQ ID NO:2 is detected by the step(s) comprising comparative genome hybridization array, amplifying the sequence, measuring the copies of the sequence using an array, and/or measuring the copies of the sequence by sequencing methods.

9. The method of claim 1, wherein at least one biological sample is obtained from the individual.

10. The method of claim 1, further comprising the step of providing one or more therapies to the individual.

11. The method of claim 10, wherein the therapy comprises vitamin supplementation, liver transplantation, carbamazepine, gene therapy, cell therapy, antisense therapy, RNA interference, or a combination thereof.

12. The method of claim 1, wherein the individual is a mammal.

13. The method of claim 1, wherein the individual is a human.

14. The method of claim 1, wherein the individual has an alpha1-antitrypsin deficiency.

15. The method of claim 1, wherein the individual has at least one copy of the ATZ variant of alpha1-antitrypsin.

16. The method of claim 1, wherein the liver disease is further defined as end stage liver disease.

17. A method for detecting a genetic signature in an individual, comprising the steps of:
(1) analyzing a region of nucleic acid from an individual suspected of having liver disease or being at risk for liver disease, wherein the nucleic acid includes polymorphism rs4567 as represented by position 101 of SEQ ID NO:1 to detect the presence or absence of an A nucleobase at position 101; and
(2) determining differential probe hybridization of an allele represented by part or all of SEQ ID NO:2.

18. The method of claim 17, wherein the method for analyzing the region of SEQ ID NO:1 in (1) comprises the steps of:
(a) amplifying a region of nucleic acid from said individual wherein the nucleic acid includes polymorphism rs4567 as represented by position 101 of SEQ ID NO:1 or its complement thereof to thereby generate an amplicon comprising said polymorphism;
(b) testing for said polymorphism rs4567 by contacting said amplicon with an oligonucleotide that selectively hybridizes to A at said position 101 of SEQ ID NO:1 or T at said complement of SEQ ID NO:1; and
(c) detecting the presence of said A or said T.

19. The method of claim 18, wherein said oligonucleotide is an allele-specific probe.

20. The method of claim 18, wherein said oligonucleotide comprises a segment of said SEQ ID NO:1 or its complement thereof, wherein said segment comprises at least 12 contiguous nucleotides in length and includes said position 101.

21. The method of claim 18, wherein said oligonucleotide is detectably labeled with a fluorescent label.

22. The method of claim 18, wherein said individual is homozygous for said A or said T.

23. The method of claim 18, wherein said individual is heterozygous for said A or said T.

24. The method of claim 18, wherein said individual has at least 3-fold differential probe hybridization at the allele represented by SEQ ID NO:2.

25. The method of claim 18, wherein the sequence represented by SEQ ID NO:2 is detected by the step(s) comprising comparative genome hybridization array, amplifying the sequence, measuring the copies of the sequence using an array, and/or measuring the copies of the sequence by sequencing methods.

26. The method of claim 18, wherein the individual is a mammal.

27. The method of claim 18, wherein the individual is a human.

28. A kit, comprising one or both of the following:
a composition for detecting polymorphism rs4567 as represented by position 101 of SEQ ID NO:1; and
a composition for detecting differential probe hybridization of SEQ ID NO:2.

29. The kit of claim 28, wherein the composition for detecting the polymorphism is an oligonucleotide, one or more primers suitable for amplifying the polymorphism, one or more sequencing reagents, or a combination thereof.

30. The kit of claim 28, wherein the composition for detecting differential probe hybridization is one or more oligonucleotides that hybridize at part or all of SEQ ID NO:2.


SEQUENCE LISTING (8 sequences)

SEQ ID NO: 1 - DNA, 201 bp, Homo sapiens
tgtgggccgaccagaggggggcttcgaggtggtccctggtactggggtgaccgagtggacagcccagggtgcagctctgcccgggctcgtgaagcctcagrtgtccccaatccaagggtctggaggggctgccgtgactccagaggcctgaggctccagggctggctctggtgtttacaagctggactcagggatcctcct

SEQ ID NO: 2 - DNA, 950 bp, Homo sapiens
aaggagtgcggcgcggcgctgtgacagggaggggaagacggggagccggacgcacgaccggcgagcacagaagggcacaggagctgctccgcgagataccagcgagctgaggcgcggggctggctgggaagtagtcccggccgggccctggccccgcccctgcagggcggagggacgcatcacgcaggggtggggaagccggatccgtgcgcagggttgctaagggtgaaacttttcattgactttgctcacttcgggccggggcgcgggaaggtgcgggttgttggctgaggcaagccgtggctcgggcgacgcggggccaggagggcaggaccgtctgccggcgacagcccataccgtctgccgttgcgcgacacgaaccaccctccgatccgccatccccgcgtcgtcgccgctagtccgcccgcccgccctcgggccccccgctgccgagcccgacctcctaagagctgaaagaaattattgagagtcatagtccatagccccctgcttcgtcccccaaccctcaacgacgaaaaggacttcggtcccctggcccggcgacgcccgggaaggaaaggagagcgacctccgccccgcgctcaggccaccctggagggagaagccgccccgcggccggtagagcgccccgccgccgcggagacccgagtgagtgagtgggggggcgtgcgggcggggggcccgccccgagacccagccggccggactacctcttccaccccgccccactcccgctttgctggactctccacgttgctgccgctccctccccgcctggccagcattttctttccccatccagcttccctccagaccccactttccccagctcctcttctcacgccatctgtgctcctggggtgcccaagagctcctttttctcccagttccaagagtggccctctcctgggtggggtgcatttcctccc

SEQ ID NO: 3 - DNA, 37 bp, Homo sapiens
cgcagggttgctaagggtgaaacttttcattgacttt

SEQ ID NO: 4 - DNA, 54 bp, Homo sapiens
cggcccgaagtgagcaaagtcaatgaaaagtttcacccttagcaaccctgcgca

SEQ ID NO: 5 - DNA, 54 bp, Homo sapiens
tgcgcagggttgctaagggtgaaacttttcattgactttgctcacttcgggccg

SEQ ID NO: 6 - DNA, 30 bp, Artificial Sequence
  Description of Artificial Sequence Synthetic probe
cggcccgaagtgagcaaagtcaatgaaaag

SEQ ID NO: 7 - DNA, 31 bp, Artificial Sequence
  Description of Artificial Sequence Synthetic probe
tgaaaagtttcacccttagcaaccctgcgca

SEQ ID NO: 8 - DNA, 31 bp, Artificial Sequence
  Description of Artificial Sequence Synthetic probe
acttttcattgactttgctcacttcgggccg